(19) United States
(12) Patent Application Publication
(10) Pub. No.: US 2024/0266025 A1
(43) Pub. Date: Aug. 8, 2024

(54) MESSAGE PROVIDING APPARATUS, MESSAGE PROVIDING METHOD, AND PROGRAM

(71) Applicant: NIPPON TELEGRAPH AND TELEPHONE CORPORATION, Tokyo (JP)

(72) Inventors: Tae SATO, Tokyo (JP); Naoto ABE, Tokyo (JP)

(73) Assignee: NIPPON TELEGRAPH AND TELEPHONE CORPORATION, Tokyo (JP)

(21) Appl. No.: 18/570,052

(22) PCT Filed: Jun. 14, 2021
(86) PCT No.: PCT/JP2021/022585

§ 371 Date: Dec. 13, 2023

(51) Int. Cl. G16H 20/70; H04L 51/02

(52) U.S. Cl. CPC G16H 20/70; H04L 51/02

(57) ABSTRACT

Conventionally, a provider has prepared a plurality of motivational messages for implementing a predetermined activity such as a health activity and provided a predetermined motivational message to the user so that the user does not get bored. An object of the present invention is to provide a motivational message aligned with a user's propensity to think, while suppressing the load on a provider of the motivational message.

The present invention is a message providing device that provides a user with a motivational message for initiating a predetermined activity, wherein a motivational message is generated by using input data indicating a current deprecated activity of the user, the effect of the deprecated activity, a recommended activity, and the effect of the recommended activity, and supplementing a component of a message stored in advance.

### TECHNICAL FIELD

The present invention relates to a message providing device, a message providing method, and a program.

### BACKGROUND ART

In preventing the onset of lifestyle-related diseases and dieting, it is important for people today to adopt the activities recommended by doctors, health professionals, and other supporters in their daily lives. For this reason, it is desirable to provide motivational support to users to implement health activities such as walking, exercise, stair climbing, low-calorie diet, low-GI diet, etc., even in situations where the supporter is out of sight.

It has conventionally been proposed to prepare a plurality of motivational messages for implementing health activities and to provide a predetermined motivational message to the user so that the user does not get bored (see NPL 1).

### CITATION LIST

### Non Patent Literature

[NPL 1] “Examining Intervention Messages in Health Activity Decision-Making: Nudges for User Cognitive Change in Daily Activity Not Performed (Progress Report)”, Tae Sato et al, Proceedings of the Poster Session of the 13th Annual Meeting of the Society for Behavioral Economics Society, Internet <URL: http://www.abef.jp/conf/2019/common/doc/poster/P07_PR0025.pdf>

### SUMMARY OF INVENTION

### Technical Problem

However, while NPL 1 provides a message policy and sample messages to motivate the user, it is a burdensome task to create a large number of motivational messages to motivate the user in advance. In addition, it is assumed that motivational messages motivate differently depending on people's tendency to think. Therefore, in order to increase the effectiveness for each individual, it is necessary to provide motivational messages that respond to users having different tendencies, not just general motivational tendencies that are effective for many people.

The present invention has been made in view of the above circumstances, and an object thereof is to provide a plurality of motivational messages aligned with different ways users are likely to be motivated, while suppressing the load on a provider of the motivational messages.

### Solution to Problem

In order to solve the above problems, the invention according to claim 1 is a message providing device that provides a user with a motivational message for initiating a predetermined activity, the message providing device including: a message generation means for generating a motivational message by using input data indicating a current deprecated activity of the user, an effect of the deprecated activity, a recommended activity, and an effect of the recommended activity, and supplementing a component of a message stored in advance.

### Advantageous Effects of Invention

As described above, the present invention brings about the effect of providing a plurality of motivational messages having different motivational tendencies, while controlling the load on the provider of the motivational messages.

### BRIEF DESCRIPTION OF DRAWINGS

FIG. 1 is a schematic diagram of a communication system.

FIG. 2 is a diagram showing an electrical hardware configuration of a message providing device.

FIG. 3 is a diagram showing an electrical hardware configuration of a communication terminal.

FIG. 4 is a functional block diagram of the message providing device.

FIG. 5 is a diagram showing a data structure of a message syntax DB.

FIG. 6 is a diagram showing a data structure of the message syntax DB.

FIG. 7 is a diagram showing a data structure of a loss expression DB.

FIG. 8 is a diagram showing a data structure of a paradoxical conjunction DB.

FIG. 9 is a diagram showing a data structure of a gain expression DB.

FIG. 10 is a diagram showing a data structure of a comparative conjunction term DB.

FIG. 11 is a diagram showing a data structure of a subjunctive conjunction term DB.

FIG. 12 is a diagram showing a data structure of a proposal expression DB.

FIG. 13 is a diagram showing a DB of thought-inducing sentence for one's body and life.

FIG. 14 is a diagram showing a second person-containing health benefit sentence DB.

FIG. 15 is a diagram showing an experience sentence DB.

FIG. 16 is a diagram showing a data structure of a DB of transmission expression in message format.

FIG. 17 is a diagram showing a data structure of a case study sentence DB.

FIG. 18 is a diagram showing a data structure of a DB of transmission expression using parallel and additional particles.

FIG. 19 is a diagram showing a data structure of an integrated effect transmission expression DB.

FIG. 20 is a diagram showing a data structure of an effect similarity system DB.

FIG. 21 shows a tree structure in a case where each group in the effect similarity system DB is a node.

FIG. 22 is a diagram showing an investigation result that led to the present invention.

FIG. 23 is a diagram showing an investigation result that led to the present invention.

FIG. 24 is a diagram showing an investigation result that led to the present invention.

FIG. 25 is a diagram showing an investigation result that led to the present invention.

FIG. 26 is a diagram showing an investigation result that led to the present invention.

FIG. 27 is a diagram showing an investigation result that led to the present invention.

FIG. 28 is a flowchart showing processing of generating a motivational message for initiating a health activity.

FIG. 29 is a flowchart showing processing of generating a motivational message for initiating a health activity.

FIG. 30 is a flowchart showing processing of generating a motivational message for initiating a health activity.

FIG. 31 is a diagram showing contents of input data.

FIG. 32 is a diagram showing an example of a message to be generated.

### DESCRIPTION OF EMBODIMENTS

Hereinafter, an embodiment of the present invention will be described based on the drawings.

### [System Configuration of Embodiment]

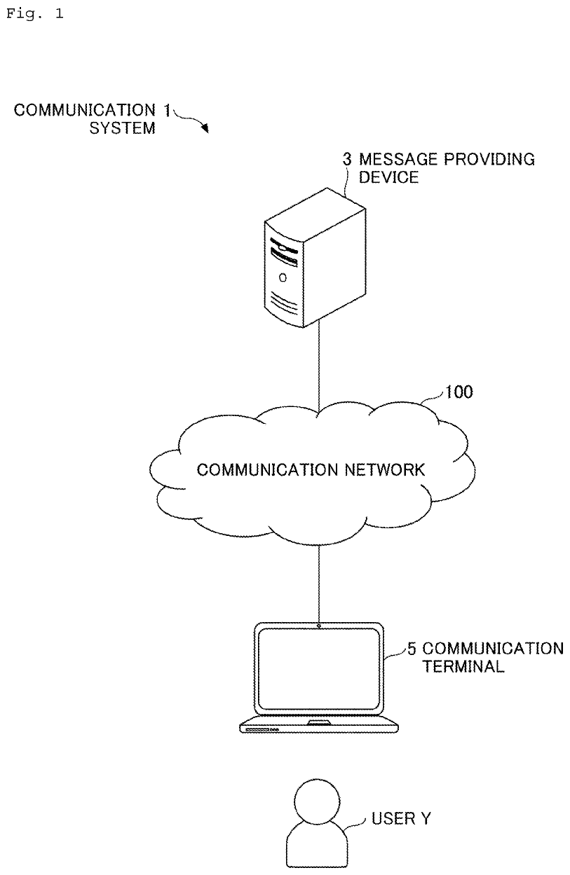

First, an overview of a configuration of a communication system according to the present embodiment will be described with reference to FIG. 1. FIG. 1 is a schematic diagram of a communication system according to the embodiment of the present invention.

As shown in FIG. 1, the communication system 1 of the present embodiment is constructed by a message providing device 3 and a communication terminal 5. The communication terminal 5 is managed and used by a user Y.

The message providing device 3 and the communication terminal 5 can communicate with each other via a communication network 100 such as the Internet. The form of connection of the communication network 100 may be either wireless or wired.

The message providing device 3 is composed of one or more computers. When the message providing device 3 is constituted by a plurality of computers, the message providing device 3 may be described as “message providing device” or “message providing system.”

The message providing device 3 is a device for providing the user Y with a motivational message for initiating a predetermined activity. Here, walking, exercising, climbing stairs, a low-calorie diet, a low-GI diet, and other health activities will be described as the predetermined activity of the user Y. The message providing device 3 outputs a motivational message. Examples of the method of outputting motivational messages include displaying or printing a motivational message on the communication terminal 5 side by transmitting the motivational message to the communication terminal 5, displaying a motivational message on a display connected to the message providing device 3, and printing a motivational message on a printer or the like connected to the message providing device 3.

The communication terminal 5 is a computer, and FIG. 1 shows a notebook-sized personal computer as an example thereof. In FIG. 1, the user Y operates the communication terminal 5.

### <Hardware Configuration>

### <Hardware Configuration of Message Providing Device)

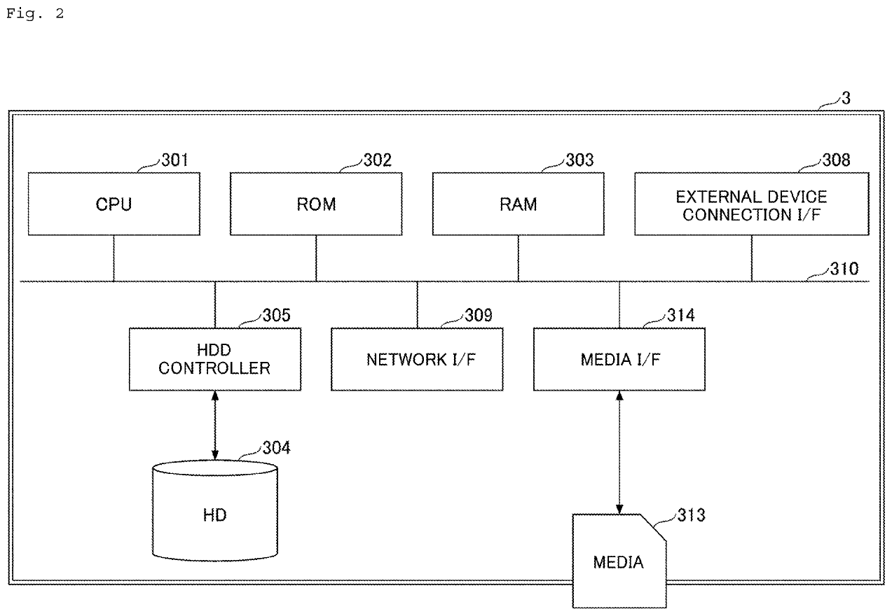

An electrical hardware configuration of the message providing device 3 will be described next with reference to FIG. 2. FIG. 2 is a diagram showing an electrical hardware configuration of the message providing device.

The message providing device 3, as a computer, includes a CPU (Central Processing Unit) 301, a ROM (Read Only Memory) 302, a RAM (Random Access Memory) 303, an HD (Hard Disk) 304, an HDD (Hard Disk Drive) controller 305, an external device connection I/F (Interface) 308, a network I/F 309, a bus line 310, and a media I/F 314.

Among them, the CPU 301 controls the operation of the entire message providing device 3. The ROM 302 stores a program used for driving the CPU 301, such as an IPL (Initial Program Loader). The RAM 303 is used as a work area of the CPU 301.

The HD 304 stores various data such as a program. The HDD controller 305 controls reading/writing of various data to/from the HD 304 according to the control of the CPU 301. In place of the HD 304 and the HDD controller 305, an SSD (Solid State Drive) and an SSD controller may be mounted.

The external device connection I/F 308 is an interface for connecting various external devices. In this case, the external device is a display, a speaker, a keyboard, a mouse, a USB (Universal Serial Bus) memory, a printer, and the like.

The network I/F 309 is an interface for realizing data communication via the communication network 100. The bus line 310 is an address bus, a data bus, or the like for electrically connecting each component such as the CPU 301 shown in FIG. 2.

Further, the media I/F 314 controls reading/writing (storing) of data to/from the recording medium 313 such as a flash memory. The recording medium 313 includes a DVD (Digital Versatile Disc), a Blu-ray Disc (registered trademark), and the like.

### <Hardware Configuration of Communication Terminal>

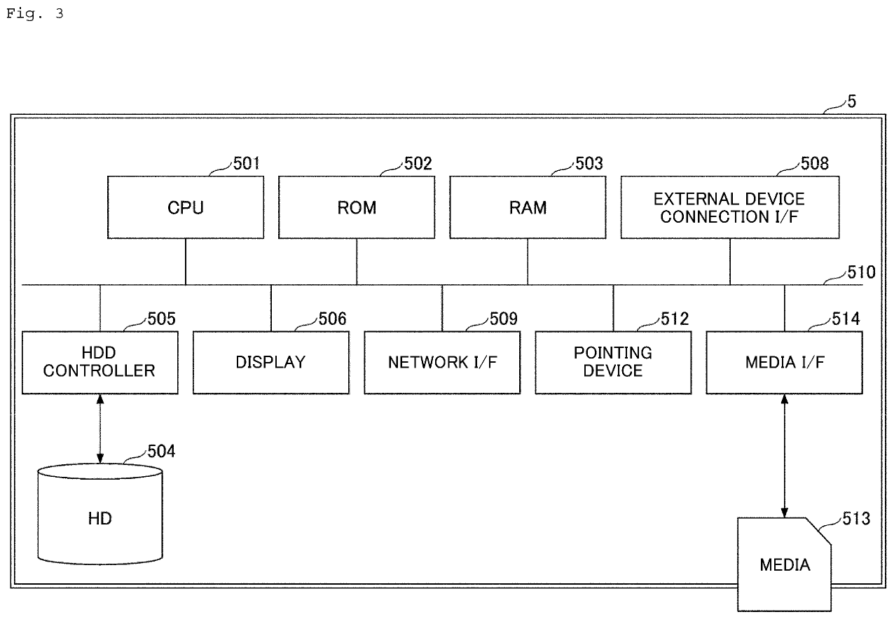

Next, an electrical hardware configuration of the communication terminal 5 will be described with reference to FIG. 3. FIG. 3 is a diagram showing an electrical hardware configuration of the communication terminal.

As shown in FIG. 3, the communication terminal 5, as a computer, includes a CPU 501, a ROM 502, a RAM 503, an HD 504, an HDD controller 505, a display 506, an external device connection I/F (Interface) 508, a network I/F 509, a bus line 510, a pointing device 512, and a media I/F 514.

Among them, the CPU 501 controls the operation of the entire communication terminal 5. The ROM 502 stores a program used for driving the CPU 501 such as an IPL. The RAM 503 is used as a work area of the CPU 501.

Various data such as programs are stored in the HD 504. The HDD controller 505 controls reading/writing of various data to/from the HD 504 according to the control of the CPU 501. In place of the HD 504 and the HDD controller 505, an SSD and an SSD controller may be mounted.

The display 506 is a type of display means such as a liquid crystal or organic EL (Electro Luminescence) for displaying various images. The external device connection I/F 508 is an interface for connecting various external devices. In this case, the external devices are a display, a speaker, a keyboard, a mouse, a USB memory, a printer, and the like.

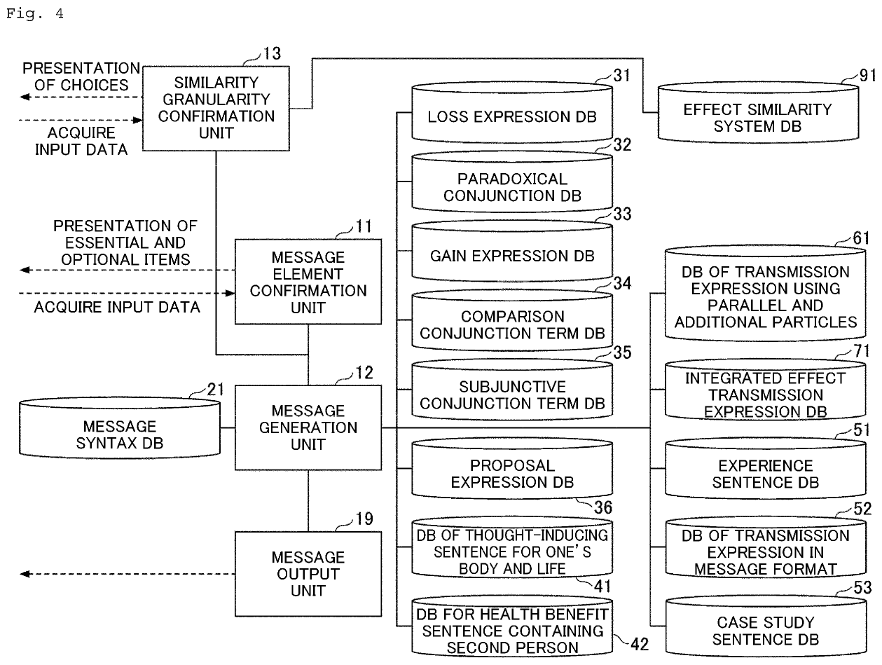

The network I/F 504 is an interface for realizing data communication via the communication network 100. The bus line 510 is an address bus, a data bus, or the like for electrically connecting each component such as the CPU 501 shown in FIG. 4.

The pointing device 512 is a type of input means for selecting and executing various instructions, selecting an object to be processed, and moving a cursor. When the user Y uses a keyboard, the function of the pointing device 512 may be turned off. The media I/F 514 controls reading/writing (storing) of data to/from the recording medium 513 such as a flash memory. The recording medium 513 includes a DVD, a Blu-ray Disc (registered trademark), and the like.

### [Functional Configuration of Message Providing Device]

Next, a functional configuration of the message providing device will be described with reference to FIG. 4. FIG. 4 is a diagram showing a functional configuration of the message providing device according to the embodiment of the present invention.

In FIG. 4, the message providing device 3 includes a message element confirmation unit 11, a message generation unit 12, a similarity granularity confirmation unit 13, and a message output unit 19. These respective units are functions realized by a command from the CPU 301 of FIG. 2 on the basis of a program.

Further, in the RAM 303 or the HD 304 shown in FIG. 2, a message syntax DB (Data Base) 21, a loss expression DB 31, a paradoxical conjunction DB 32, a gain expression DB 33, a comparative conjunction term DB 34, a subjunctive conjunction term DB 35, a proposal expression DB 36, a DB 41 of thought-inducing sentence for one's body and life, a second person-containing health benefit sentence DB 42, an experience sentence DB 51, a DB 52 of transmission expression in message format, a case study sentence DB 53, a DB 61 of transmission expression using parallel and additional particles, and an integrated effect transmission expression DB 71 are constructed. Each of these DBs is constituted by a table as shown below. In the present embodiment, the information managed in each DB is combined as a component, to generate a motivational message for initiating the execution of a health activity.

### <DB Configuration>

### (Message Syntax DB)

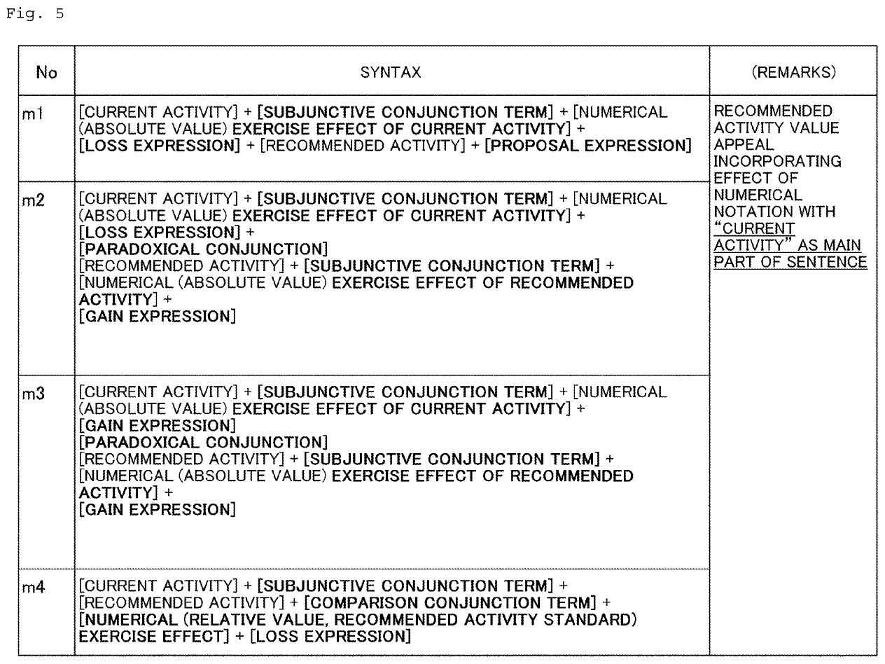

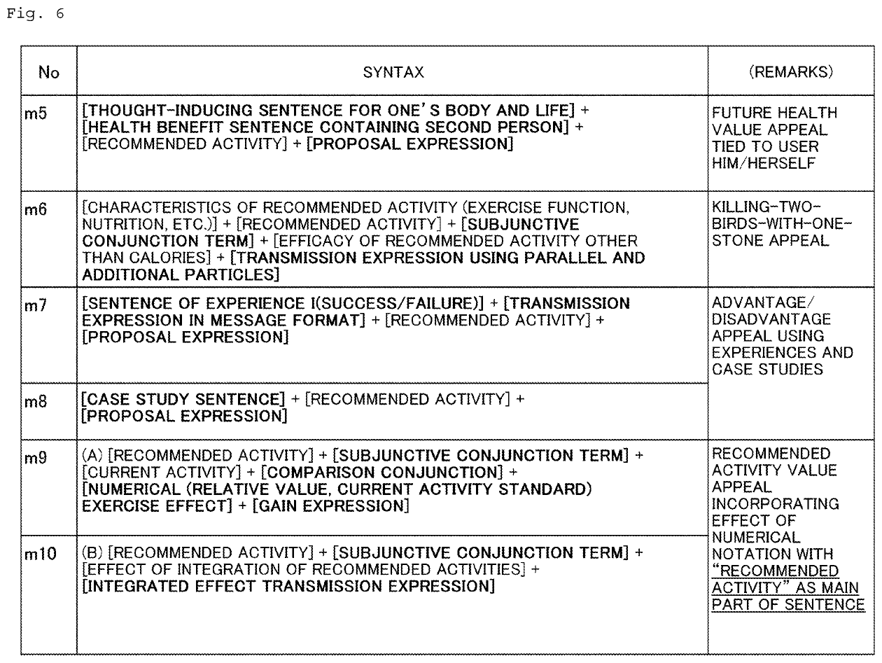

FIGS. 5 and 6 are data configuration diagrams of the message syntax DB. In the message syntax DB 21, syntax and remarks are associated and managed for each message number for identifying the message.

### (Loss Expression DB)

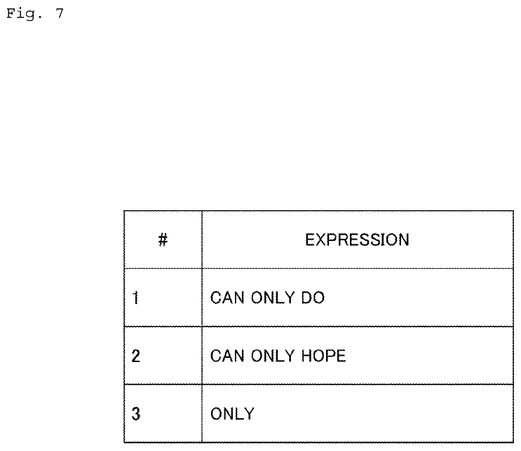

FIG. 7 is a data configuration diagram of the loss expression DB. In the loss expression DB 31, information indicating the expression of loss is managed for each reference number.

### (Paradoxical Conjunction DB)

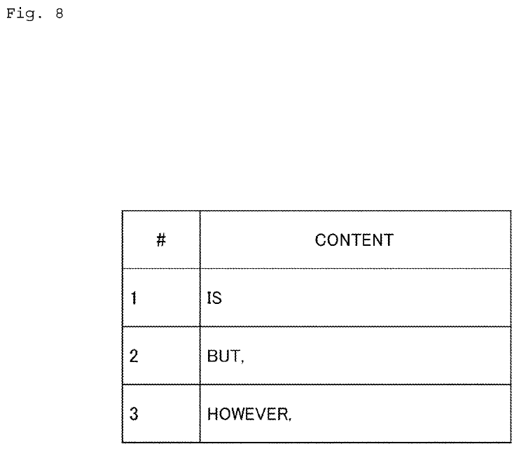

FIG. 8 is a data configuration diagram of the paradoxical conjunction DB 32. In the paradoxical conjunction DB 32, information indicating the content of a paradoxical conjunction is managed for each reference number.

### (Gain Expression DB)

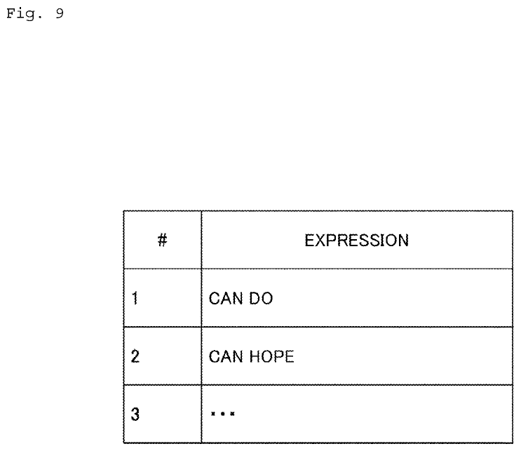

FIG. 9 is a data configuration diagram of the gain expression DB 33. In the gain expression DB 33, information indicating the expression of a gain is managed for each reference number.

### (Comparative Conjunction Term DB)

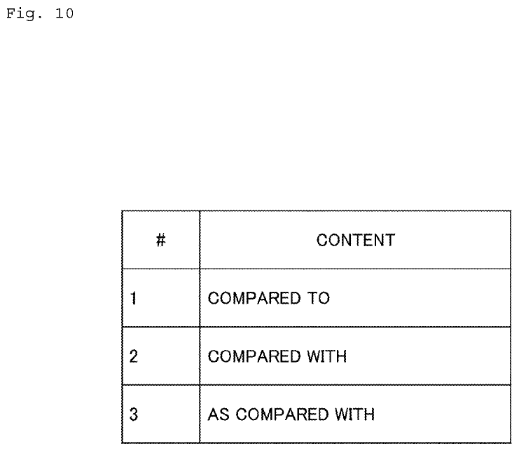

FIG. 10 is a data configuration diagram of the comparison conjunction term DB. In the comparison conjunction term DB 34, information indicating the content of a comparison conjunction term is managed for each reference number.

### (Subjunctive Conjunction Term DB)

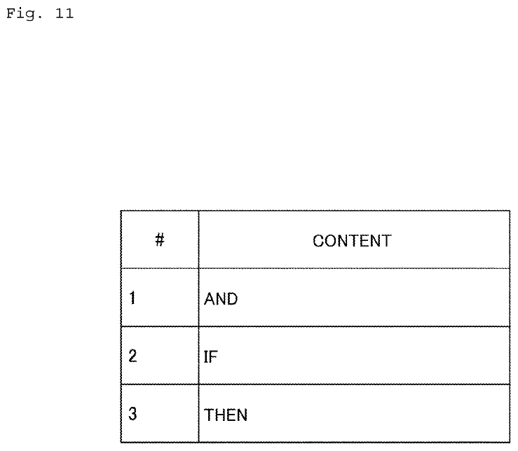

FIG. 11 is a data configuration diagram of the subjunctive conjunction term DB. In the subjunctive conjunction term DB 35, information indicating the content of a subjunctive conjunction term is managed for each reference number.

### (Proposal Expression DB)

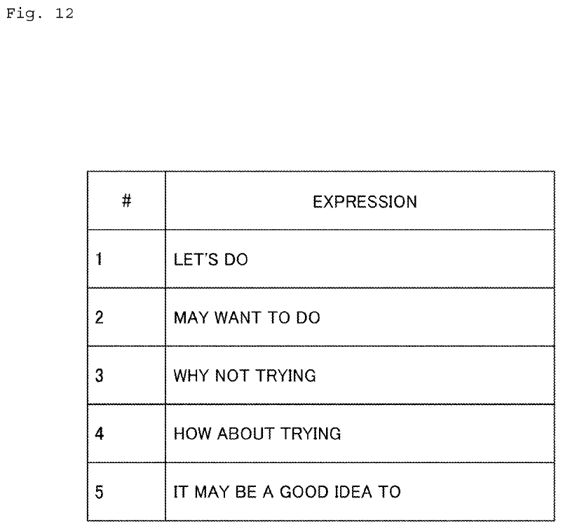

FIG. 12 is a data configuration diagram of the proposal expression DB. In the proposal expression DB 36, information indicating a proposal expression is managed for each reference number.

### (DB of Thought-Inducing Sentence for One's Body and Life)

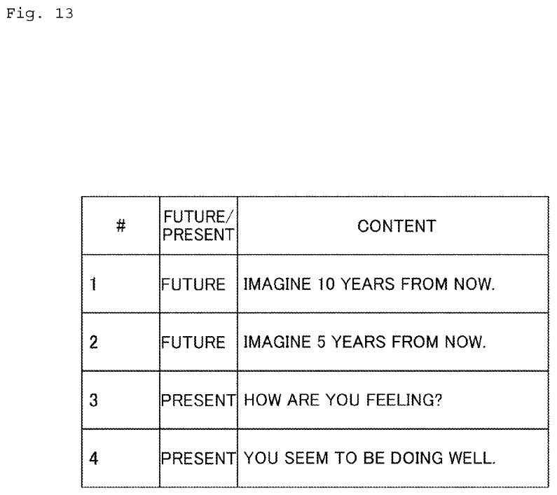

FIG. 13 is a data configuration diagram of the DB of thought-inducing sentence for one's body and life. In the DB 41 of thought-inducing sentence for one's body and life, reference numbers, time information indicating the future or present, and information indicating the content of a thought-inducing sentence for one's body and life, are associated and managed.

### (Second Person-Containing Health Benefit Sentence DB)

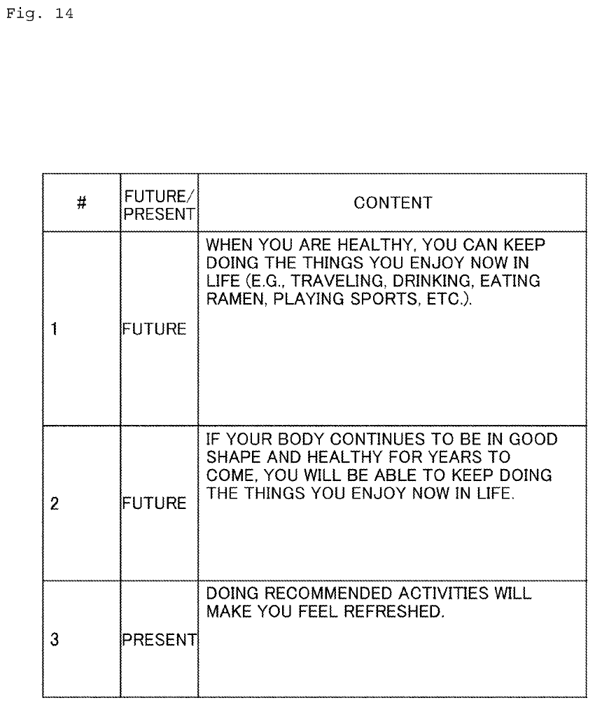

FIG. 14 is a data configuration diagram of the second person-containing health benefit sentence DB. In the second person-containing health benefit sentence DB 42, reference numbers, time information indicating the future or present, and information indicating the content of a second person-containing health benefit sentence, are associated and managed.

### (Experience Sentence DB)

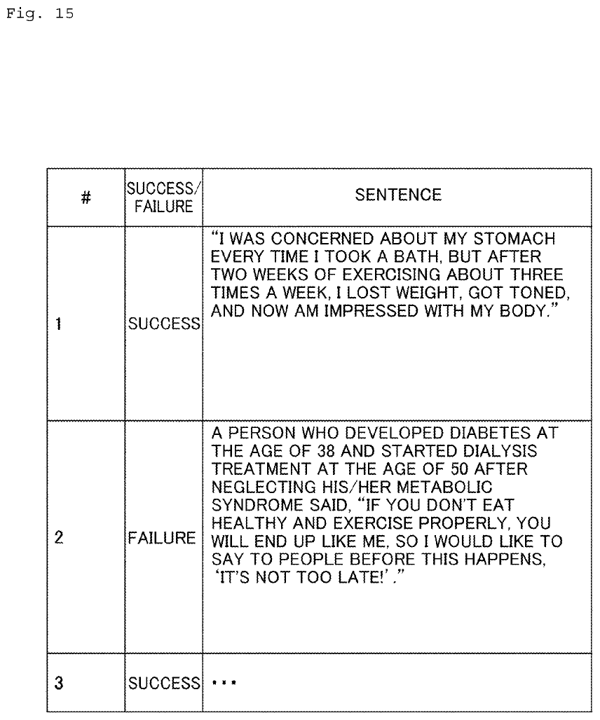

FIG. 15 is a data configuration diagram of the experience sentence DB. In the experience sentence DB 51, reference numbers, information indicating a success or failure of a health activity, and information indicating a sentence of a success or failure story of a health activity, are associated and managed.

### (DB of Transmission Expression in Message Format)

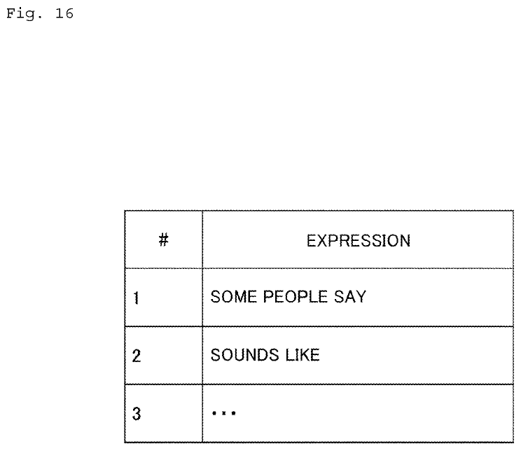

FIG. 16 is a data configuration diagram of the DB of transmission expression in message format. In the DB 52 of transmission expression in message format, information indicating the content of a transmission expression in message format is managed for each reference number.

### (Case Study Sentence DB)

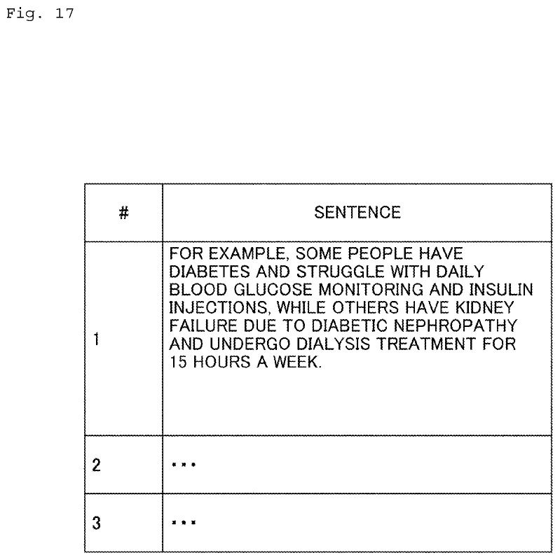

FIG. 17 is a data configuration diagram of the case study sentence DB. In the case study sentence DB 53, information indicating the content of a case study sentence is managed for each reference number.

### (DB of Transmission Expression Using Parallel and Additional Particles)

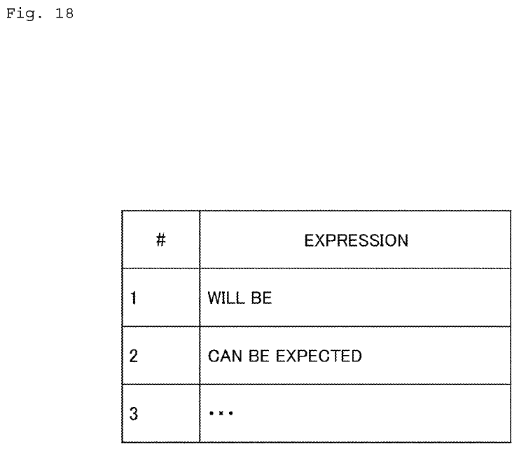

FIG. 18 is a data configuration diagram of the DB of transmission expression using parallel and additional particles. In the DB 61 of transmission expression using parallel and additional particles, information indicating the content of a transmission expression using parallel and additional particles is managed for each reference number.

### (Integrated Effect Transmission Expression DB)

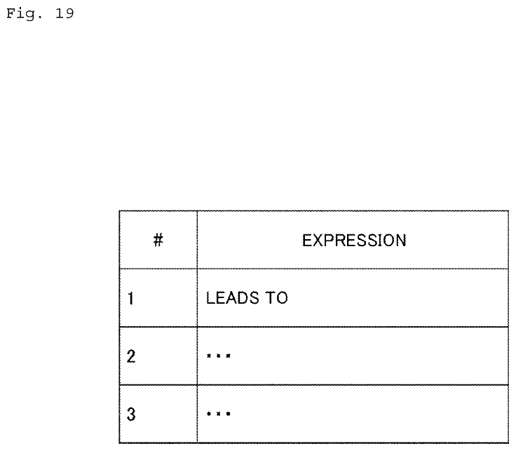

FIG. 19 is a data configuration diagram of the integrated effect transmission expression DB. In the integrated effect transmission expression DB 71, information indicating the content of an integrated effect transmission expression is managed for each reference number.

### (Effect Similarity System DB)

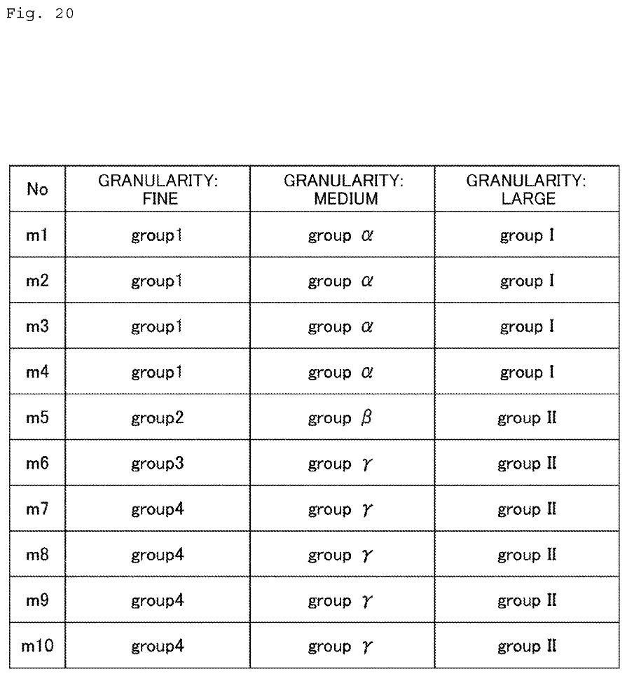

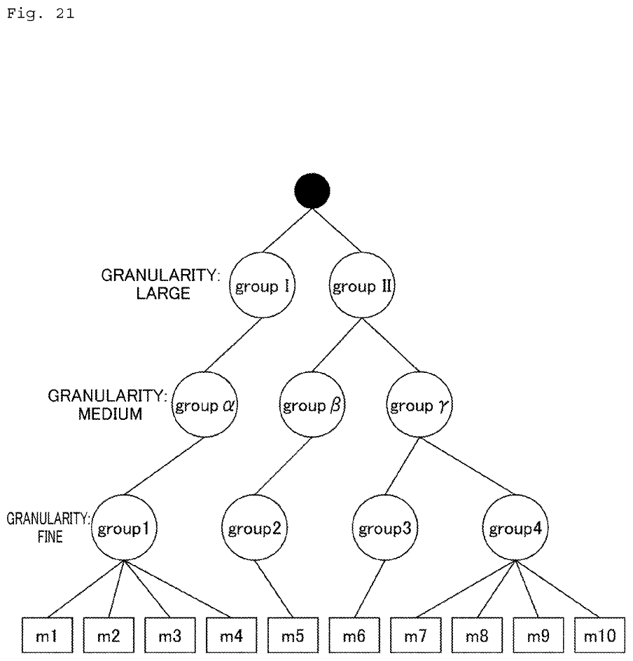

FIG. 20 is a data configuration diagram of an effect similarity system DB. An effect similarity system DB 91 shows a case where the granularity of similarity is defined in three levels (large, medium, fine). In the effect similarity system DB 91, for respective message numbers for identifying messages, group numbers indicating groups when the granularity is “fine” (fine), “medium” (medium), and “large” (large) are associated and managed. FIG. 21 is a diagram showing a tree structure in a case where these groups are taken as nodes.

### <Each Functional Configuration>

Next, each functional configuration of the message providing device will be described with reference to FIG. 4.

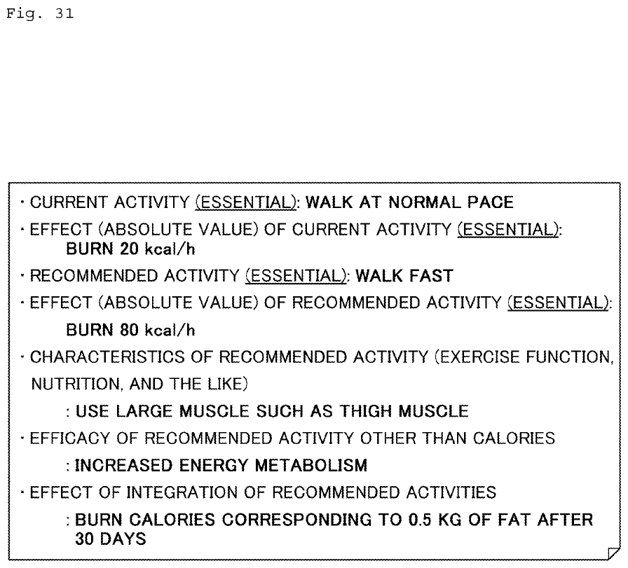

The message element confirmation unit 11 presents essential items and optional items to the user Y, thereby acquiring input data (see FIG. 31) from the user Y and checking the input of a message element.

The message generation unit 12 generates a motivational message to be provided to the user Y or the like. In this case, the message generation unit 12 refers to the message syntax DB 21 and inserts the corresponding input data and components acquired from the respective DBs 31 to 36, 41, 42, 51 to 53, 61, 71 (hereafter referred to as “DB 31 or the like”) into the corresponding part of the message syntax, to generate the message. When there are a plurality of components acquired from each DB 31 or the like by the message generation unit 12, the message generation unit 12 combines the components in total. If the required number of messages is determined, messages can be created up to that number.

The similarity granularity confirmation unit 13 imparts a similarity label to each message in accordance with an input related to the granularity of the similarity of the message. The granularity of the similarity is set in advance by a system user (manager) of the message providing device 3.

The message output unit 19 outputs the message generated by the message generation unit 12 from the message providing device 3 as a motivational message for initiating a health activity.

### <Processing or Operation of Embodiment>

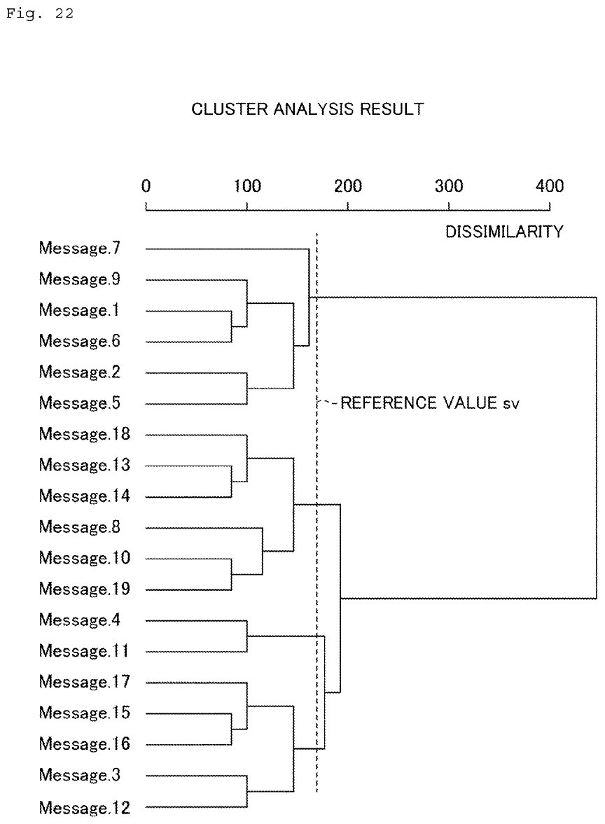

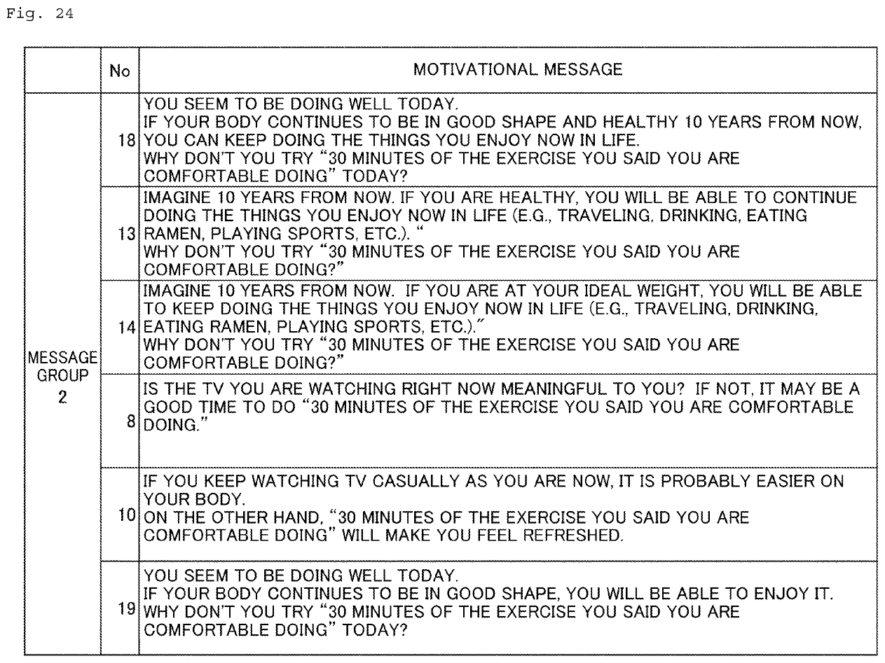

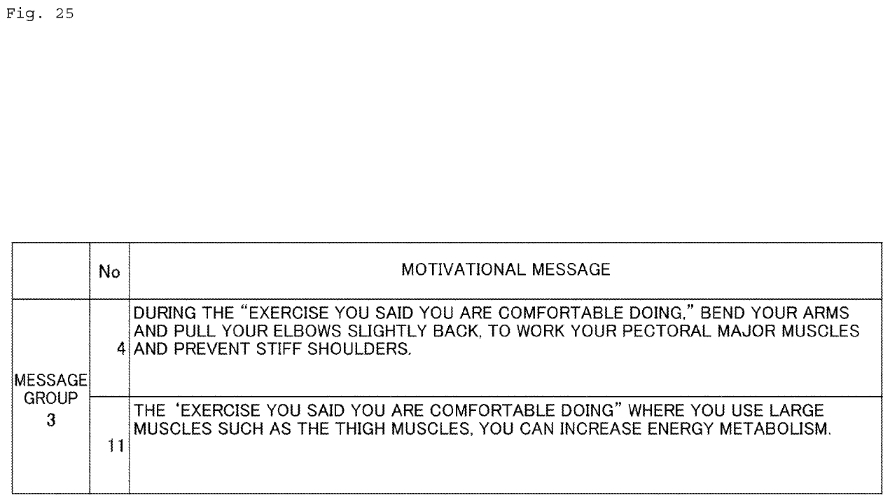

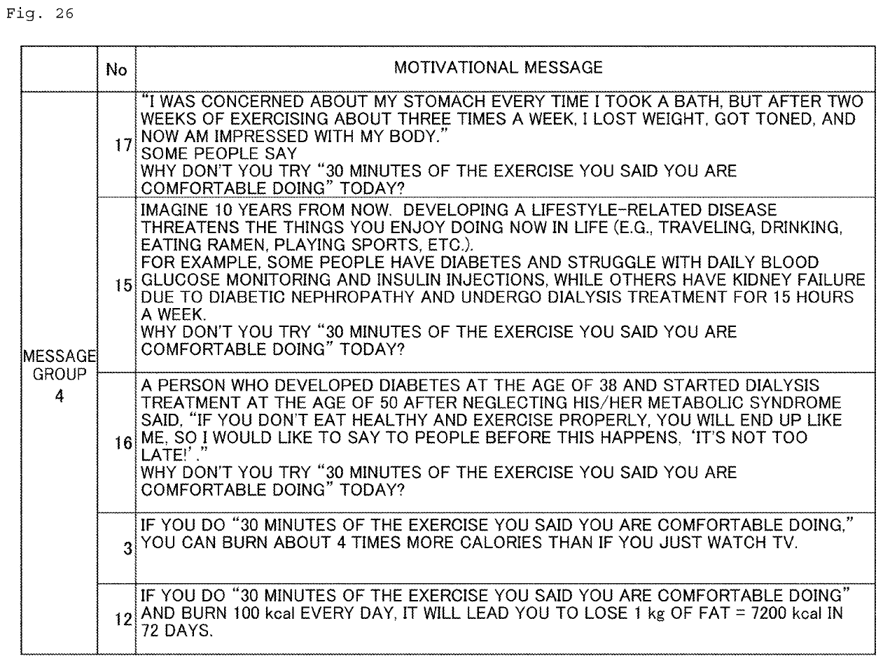

Hereinafter, the processing or operation of the present embodiment will be described in detail with reference to FIGS. 22 to 32.

<Investigation Results that LED to the Present Invention>

First, investigation results that led to the present invention will be described with reference to FIGS. 22 to 27.

We conducted a survey to ascertain whether or not people who read motivational messages are motivated to initiate health activities. More information on this is disclosed in the reference (“Examining Intervention Messages in Health Activity Decision Making: Testing the Effectiveness of Motivational Nudge Messages,” 14th Annual Meeting of the Behavioral Economics Society, http://www.abef.jp/conf/2020_archive/common/doc/program/P01.pdf).

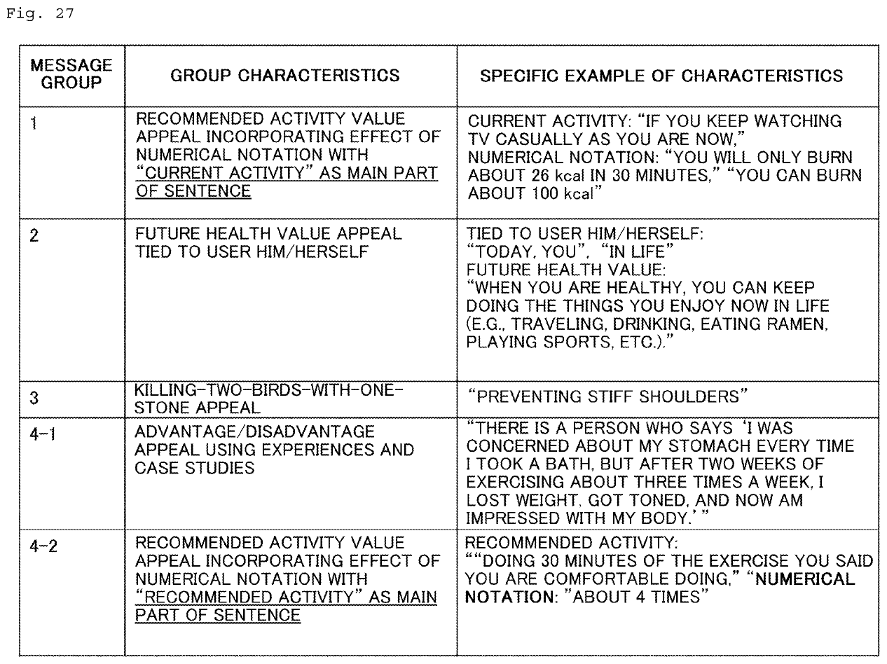

As a result of cluster analysis on the similarity of the motivating effect of a motivational message using the questionnaire results, the results shown in FIGS. 22 to 26 (four message groups) were obtained, and the characteristics of the message were seen in each group, as shown in FIG. 27. The present invention is based on these investigation results.

FIG. 22 is a diagram showing the results of hierarchical cluster analysis. The hierarchical cluster analysis is a method in which clusters are formed by starting from the most similar combinations, wherein the process can be expressed as a hierarchy, resulting in a tree diagram (dendrogram) such as the one shown in FIG. 22. Here, the Ward Method is used as an inter-cluster distance measurement method. The upper part of FIG. 22 shows the dissimilarity between two clusters. For example, messages No. 1 and No. 6 are merged at a dissimilarity position of approximately 80. The smaller the value of dissimilarity, the closer (more similar) the messages are to each other.

As an example, the case where the dissimilarity is approximately 175 is set as a reference value sv is shown. The reference value sv is a value obtained when each message is divided into four groups.

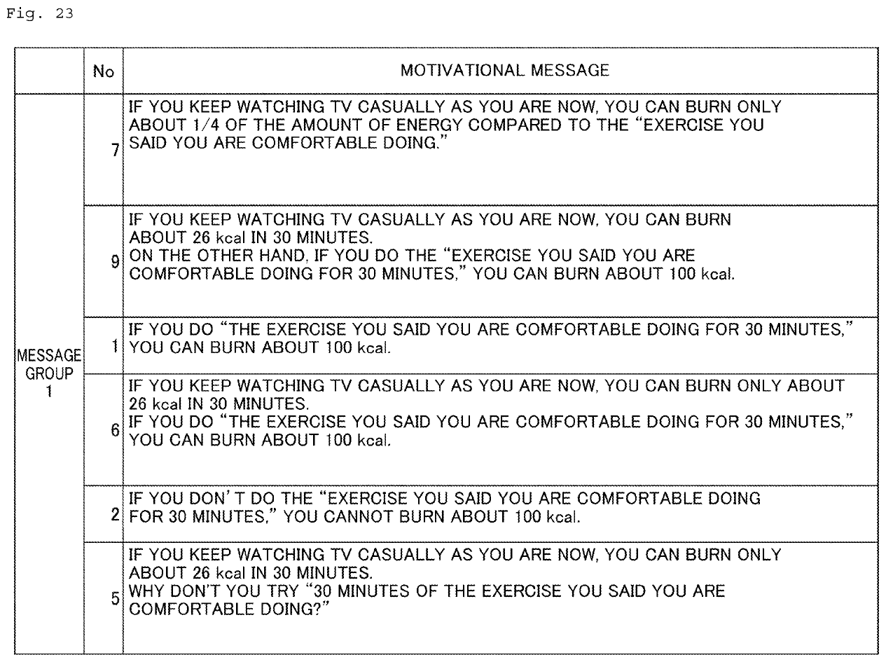

FIGS. 23 to 27 show information indicating message identification numbers for identifying messages and contents of motivational messages about health activities for each message group classified by the hierarchical cluster analysis. FIGS. 23 to 27 show motivational messages in the case where the messages are divided into four groups by the hierarchical cluster analysis shown in FIG. 22.

FIG. 27 is a diagram showing the characteristics of a group and a specific example of the characteristics for each message group number for identifying the message group. As shown in FIG. 27, message characteristics were expressed in each group, with message group No. 4 showing two characteristics.

As shown in FIG. 20, the group characteristics are broadly classified into the following four categories.
Message group No. 1: A group of messages that promote the value of recommended activities incorporating the effect of numerical notation with “current activity” as the main part of the sentence.
Message group No. 2: A group of messages that promote future health values tied to the user.
Message group No. 3: A group of messages that promote additional benefits in addition to the original purpose (killing-two-birds-with-one-stone appeal)
Message group No. 4: (4-1) A group of messages appealing to the benefits and disadvantage using experiences and examples, and (4-2) a group of messages appealing to the value of recommended activities incorporating the effect of numerical notation with “recommended activities” as the main part of the sentence.
   The present embodiment is based on the investigation results described above.

### <Processing or Operation of the Present Invention>

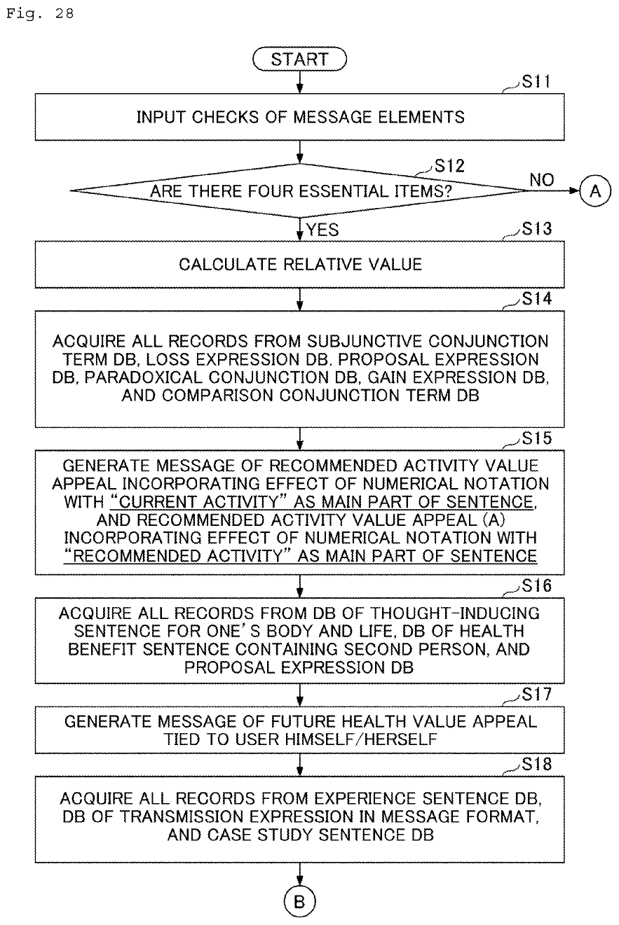

Next, the processing or operation of the present invention based on the above-mentioned investigation results will be described with reference to FIGS. 28 to 32. FIGS. 28 to 30 are flow charts showing the processing of generating a motivational message for initiating a health activity. FIG. 31 is a diagram showing the contents of input data input to the message providing device 3. Of this input data, the “current activity” is assumed to be obtained by a question that the user Y separately asks a person (e.g., user X) who promotes health activity, or by observing the user X, or by detecting his/her activity using a sensor. The other items are input by the user Y. When the user Y inputs the data, the data may be presented in the form of a question, to obtain the input data.

First, as shown in FIG. 28, the message element confirmation unit 11 presents, to the user Y, essential items and arbitrary items of input related to a health activity, and performs input checks of motivational message elements with respect to the input data obtained from the user Y (S11).

In FIG. 31, as the essential items, four items of “current activity (deprecated activity),” “effect (absolute value) of current activity,” “recommended activity,” and “effect (absolute value) of recommended activity” are shown. Further, as the arbitrary items, three items of “characteristics of recommended activity (motor function, nutrition, etc.),” “efficacy of recommended activity other than calories,” and “effect by integration of recommended activities” are shown.

In a case where there are the foregoing four essential items by the input checks by the message element confirmation unit 11 (S12; YES), the message element confirmation unit 11 calculates an absolute value (S13). More specifically, the message element confirmation unit 11 calculates the “numerical (relative value, current activity standard) exercise effect” and “[numerical (relative value, recommended activity standard) exercise effect]” by using the “numerical (absolute value) exercise effect of the current activity (deprecated activity)” and “numerical (absolute value) exercise effect of the recommended activity.” For example, in case of the input data shown in FIG. 31, the following values (absolute values) are calculated from:
The effect of the current activity (absolute value): 20 kcal/h consumption
The effect of recommended activity (absolute value): 80 kcal/h consumption
Exercise effect of numeral (relative value, current activity standard): 4 times calorie consumption
Exercise effect of numeral (relative value, recommended activity standard): ¼ of calorie consumption
   In a case where there are no abovementioned essential four items by the input checks performed in step S11 (S12; NO), the processing of S13 to S31 is not performed.

Then, the message generation unit 12 acquires all records from the loss expression DB 31, the paradoxical conjunction DB 32, the gain expression DB 33, comparative conjunction DB 34, the subjunctive conjunction term DB 35, and the proposal expression DB 36 (S14).

Next, the message generation unit 12 generates a message of recommended activity value appeal (A) incorporating the effect of numerical notation with the “current activity” as the main part of the sentence, and a message of recommended activity value appeal (B) incorporating the effect of numerical notation with the “recommended activity” as the main part of the sentence (S15).

Then, the message generation unit 12 acquires all records from the DB 41 of thought-inducing sentence for one's body and life, the second person-containing health benefit sentence DB 42, and the proposal expression DB 36 (S16). When the data of the DB 41 of thought-inducing sentence for one's body and life and the data of the second person-containing health benefit sentence DB 42 are inserted, the combination is possible only when the “future/present” flags match.

Next, the message generation unit 12 generates a message of a future health appeal value tied to the user him/herself (S17). Further, the message generation unit 12 acquires all records from the experience sentence DB 51, the DB 52 of transmission expression in message format, the case study sentence DB 53, and the proposal expression DB 36 (S18).

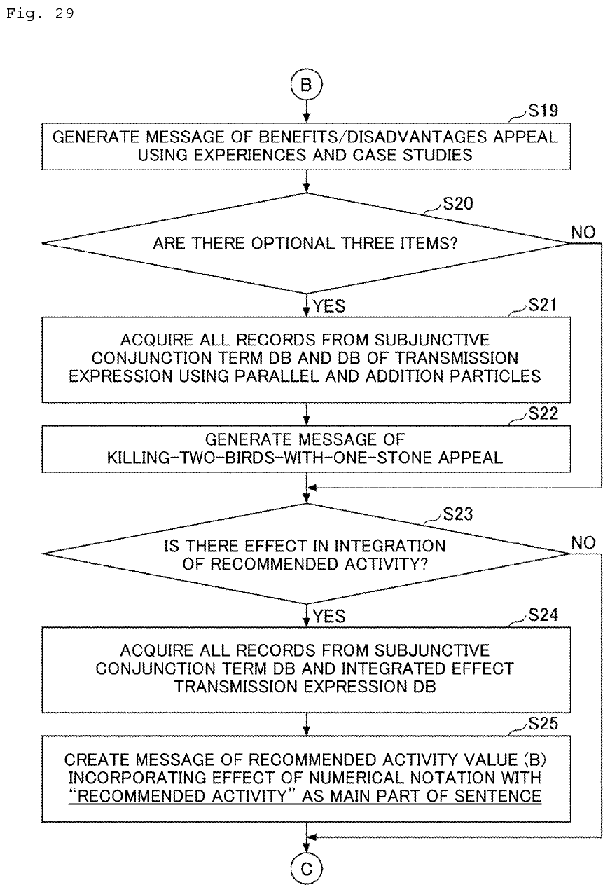

Subsequently, as shown in FIG. 29, the message generation unit 12 generates a message appealing to the benefits and disadvantages using experiences and examples (S19). The advantage appeal is to convey reasons why the user Y benefits from the person who promotes the health activity (user X). The disadvantage appeal is to convey reasons why the user Y will lose (harm) to the person who promotes the health activity (user X).

Next, the message generation unit 12 determines whether or not three optional items (the characteristics of the recommended activity, the efficacy of the recommended activity other than calories, and the effects of the integration of recommended activities) are described in the input data (S20). If these items are described in the input data (S20; YES), the message generation unit 12 acquires all records from the subjunctive conjunction term DB and DB 61 of transmission expression using parallel and additional particles (S21).

Next, the message generation unit 12 generates a killing-two-birds-with-one-stone appeal message (S22).

On the other hand, in step S20, if the three optional items are not described (S20; NO), the processing of steps S21 and S22 is omitted.

Next, the message generation unit 12 determines whether or not there is an effect by the integration of recommended activities (S23). If there is an effect (S23: YES), the message generation unit 12 acquires all records from the subjunctive conjunction term DB 35 and then integrated effect transmission expression DB 71 (S24).

Next, the message generation unit 12 creates the message of recommended activity value appeal (B) incorporating the effect of numerical notation with the “recommended activity” as the main part of the sentence (S25).

On the other hand, if there is no effect in step S23 (S23; NO), the processing of steps S24 and S25 is omitted.

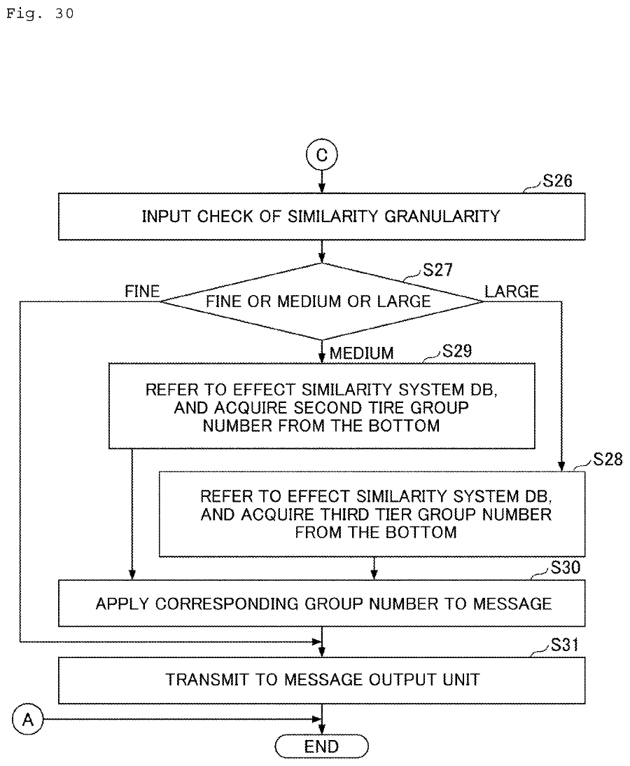

Next, as shown in FIG. 30, the similarity granularity confirmation unit 13 presents choices to the user Y, acquires the input data from the user Y, and performs the input checks on similarity granularity (S26). When the similarity granularity is large (S27; large), the similarity granularity confirmation unit 13 refers to the effect similarity system DB 91 and acquires the third tier group number from the bottom (S28). When the similarity granularity is medium (S27; medium), the similarity granularity confirmation unit 13 refers to the effect similarity system DB 91 and acquires the second tier group number from the bottom (S29). Then, after steps S28 and S29, the similarity granularity confirmation unit 13 assigns the acquired corresponding group numbers as the similarity labels, to the message (S30).

When the similarity granularity is fine (S27; fine), steps S28 to S30 are omitted.

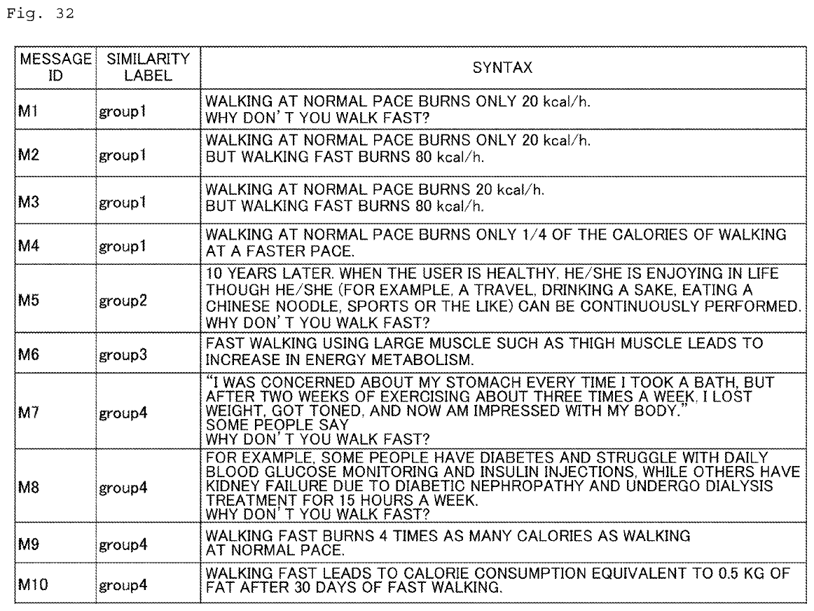

Then, the message generation unit 12 transmits the generated message to the message output unit 19 (S31). FIG. 32 is a diagram showing an example of the message generated by the message generation unit 12. The message consists of a message ID (Identification), a similarity label, and a message syntax (content).

As described above, the message output unit 19 can output the message generated by the message generation unit 12 as a motivational message for initiating a health activity.

### <Effects of Embodiment>

As described above, according to the present embodiment, the message generation unit 12 generates a plurality of different messages by using the “current activity (deprecated activity),” “numerical (absolute value) exercise effect of current activity (deprecated activity),” “recommended activity,” and “numerical (absolute value) exercise effect of deprecated activity” as the input and by supplementing particles, conjunctions and the like stored in advance. Then, the similarity granularity confirmation unit 13 assigns a similarity label to each message in response to an input related to the similarity granularity of the message.

Furthermore, the message generation unit 12 generates a plurality of different messages by supplementing particles, conjunctions and the like stored in advance, in response to the presence/absence of an input of the “characteristics of the recommended activity,” “efficacy of the recommended activity other than calories,” and “effect by integration of recommended activities.” Then, the similarity granularity confirmation unit 13 assigns a similarity label to each message in response to an input related to the similarity granularity of the message.

Thus, the message providing device 3 can create a group of messages with similar motivational effects on the user Y.

Therefore, for example, the message providing device 3 can use a log indicating that the user Y has been motivated by one message, to provide another motivational message that has been motivational, or can acquire, in advance, the user characteristics related to motivational tendencies, to select a motivational message corresponding to the user characteristics.

Also, the system user (manager) (user Y) of the message providing device 3 can set the similarity granularity. Therefore, for example, when using the message providing device which is assumed to be used less frequently by the user X, the similarity granularity can be made coarse, and conversely, when using the message providing device which is assumed to be used frequently, the similarity granularity can be made fine.

### <Additional Notes>

The present invention is not limited to the embodiment described above, but may be configured or processed (operated) as described below.

(1) In the foregoing embodiment, a health activity is described as the predetermined activity initiated by a motivational message, but the present invention is not limited thereto. Examples of the predetermined activity may include a learning activity such as a homework, a preliminary study, and a review for school. The predetermined activity may also be a job hunting (activity) by students, such as preparation of entry sheets and visiting companies.

(2) The message providing device 3 according to the present invention can also be realized by a computer and a program, but can also be recorded on a recording medium or provided through a communication network.

(3) In the foregoing embodiment, a notebook-sized personal computer is illustrated as an example of the communication terminal 5, but the present invention is not limited thereto, and, for example, a desktop personal computer, a tablet terminal, a smart phone, a smart watch, a car navigation device, a refrigerator, a microwave and the like may be used as the communication terminal 5.

(4) Each CPU 301, 501 may be a single as well as multiple.

(5) A neural network (Neural Network) may be used in at least one of the types of processing of the message generation unit 12.

### REFERENCE SIGNS LIST

1 Communication system
3 Message providing device
5 Communication terminal
11 Message element confirmation unit
12 Message generation unit
13 Similarity granularity confirmation unit
19 Message output unit
21 Message syntax DB
31 Loss expression DB
32 Paradoxical conjunction DB
33 Gain expression DB
34 Comparison conjunction term 34
35 Subjunctive conjunction term DB
36 Proposal expression DB
41 DB of thought-inducing sentence for one's body and life
42 DB of second person-containing health benefit sentence
51 Experience sentence DB
52 DB of transmission expression in message format
53 Case study sentence DB
61 DB of transmission expression using parallel and additional particles
71 Integrated effect transmission expression DB

1. A device for providing a user with a motivational message for initiating a predetermined activity, the device comprising:
receiving input data, wherein the input data indicates a deprecated activity of the user, an effect of the deprecated activity, the predetermined activity as a recommended activity, and an effect of the recommended activity; and
generating, based on the input data, the motivational message by supplementing a component of a message stored in advance.

2. The device according to claim 1, the processor further configured to execute operations comprising:
determining a similarity viscosity of the input data; and
attaching a similarity label to the motivational message based on the determined similarity viscosity.

3. The device according to claim 1, wherein the generating further comprises generating the motivational message by supplementing a component of a message stored in advance, in accordance with:
characteristics of the recommended activity,
an efficacy of the recommended activity other than calories, and
an effect of integration of recommended activities are indicated by the input data.

4. The device according to claim 1, further comprising:
outputting the generated motivational message.

5. The device according to claim 1, wherein the predetermined activity represents at least one of: a healthcare activity, a learning activity, or a job hunting of the user.

6. The device according to claim 1, wherein the generating the motivational message further comprises generating, by a neural network, the motivational message.

7. A method providing a user with a motivational message for initiating a predetermined activity, comprising:
receiving input data, wherein the input data indicates a deprecated activity of the user, an effect of the deprecated activity, the predetermined activity as a recommended activity, and an effect of the recommended activity; and
generating, based on the input data, the motivational message by supplementing a component of a message stored in advance.

8. A computer-readable non-transitory recording medium storing a computer-executable program instructions that when executed by a processor cause a computer system to execute operations comprising:
receiving input data, wherein the input data indicates a deprecated activity of a user, an effect of the deprecated activity, a predetermined activity as a recommended activity, and an effect of the recommended activity; and
generating, based on the input data, a motivational message by supplementing a component of a message stored in advance.

9. The device according to claim 2, wherein the generating further comprises generating the motivational message by supplementing a component of a message stored in advance, in accordance with:
characteristics of the recommended activity,
an efficacy of the recommended activity other than calories, and
an effect of integration of recommended activities are indicated by the input data.

10. The method according to claim 7, further comprising:
determining a similarity viscosity of the input data; and
attaching a similarity label to the motivational message based on the determined similarity viscosity.

11. The method according to claim 7, wherein the generating further comprises generating the motivational message by supplementing a component of a message stored in advance, in accordance with:
whether or not characteristics of the recommended activity,
an efficacy of the recommended activity other than calories, and
an effect of integration of recommended activities are indicated by the input data.

12. The method according to claim 7, further comprising:
outputting the generated motivational message.

13. The method according to claim 7, wherein the predetermined activity represents at least one of: a healthcare activity, a learning activity, or a job hunting of the user.

14. The method according to claim 7, wherein the generating the motivational message further comprises generating, by a neural network, the motivational message.

15. The method according to claim 10, wherein the generating further comprises generating the motivational message by supplementing a component of a message stored in advance, in accordance with:
characteristics of the recommended activity,
an efficacy of the recommended activity other than calories, and
an effect of integration of recommended activities are indicated by the input data.

16. The computer-readable non-transitory recording medium according to claim 8, the computer-executable program instructions when executed further causing the computer system to execute operations comprising:
determining a similarity viscosity of the input data; and
attaching a similarity label to the motivational message based on the determined similarity viscosity.

17. The computer-readable non-transitory recording medium according to claim 8, wherein the generating further comprises generating the motivational message by supplementing a component of a message stored in advance, in accordance with:
characteristics of the recommended activity,
an efficacy of the recommended activity other than calories, and
an effect of integration of recommended activities are indicated by the input data.

18. The computer-readable non-transitory recording medium according to claim 8, wherein the predetermined activity represents at least one of: a healthcare activity, a learning activity, or a job hunting of the user.

19. The computer-readable non-transitory recording medium according to claim 8, wherein the generating the motivational message further comprises generating, by a neural network, the motivational message.

20. The computer-readable non-transitory recording medium according to claim 16, wherein the generating further comprises generating the motivational message by supplementing a component of a message stored in advance, in accordance with:
characteristics of the recommended activity,
an efficacy of the recommended activity other than calories, and
an effect of integration of recommended activities are indicated by the input data.
